(19) United States
(12) Patent Application Publication
(10) Pub. No.: US 2009/0062702 A1
(43) Pub. Date: Mar. 5, 2009

(54) Thermal device for application to the spine

(76) Inventors: Marcy Elizabeth Sojka, Neenah, WI (US); Sarah Lee Christoffel, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/897,694

(22) Filed: Aug. 31, 2007

(51) Int. Cl. A61F 5/00

(52) U.S. Cl. 602/2

(57) ABSTRACT

A thermal wrap that extends down at least a portion of a user's spine. Thermal element(s) may be placed along the length of the thermal wrap to warm the spine region. Optionally, thermal element(s) are located in wing portions that may contact the user's upper back.

### BACKGROUND OF THE INVENTION

The present invention relates to thermal sources for relaxation of the body, and more particularly to wraps wherein thermal energy is applied to specific areas of the spine. Even more particularly, the present invention relates to body wraps having position maintenance features.

Upper back, neck, and shoulder pain tension may be associated with stress, poor posture, and possibly, disease. One common way to reduce such tension is to visit a spa for heat therapy, in particular, hot stone therapy. Hot stone therapy involves the placement of warm, smooth stones on various regions that can experience tension in an attempt to relax the muscles, such as along the spine. However, it is not always convenient or affordable to visit a spa. What is needed is an inexpensive disposable thermal source that can be a substitute for hot stone therapy available at a spa.

### SUMMARY OF THE INVENTION

In one aspect of the present invention there is a thermal wrap for a user's spine constructed with an elongated piece of flexible material defining a central body portion having a first end and a second end, and a first wing and a second wing portion extending outwardly from the first end. The first thermal element has an exothermic chemistry, and is disposed on the central body portion. The central body portion and the first thermal element are adapted to be disposed on the user's spine during use.

In another aspect of the invention, there is a kit for providing heat to a user's spine. The kit includes an elongated piece of flexible material defining a central body portion having a first end and a second end, with a first wing portion and a second wing portion extending outwardly from the first end. A thermal element having an exothermic chemistry is adapted to be disposed on the central body portion. Together, the central body portion and the thermal element are adapted to be disposed on the user's spine during use.

Advantages of the present invention will be apparent from a review of the following disclosure. In the description, reference is made to the accompanying drawings which form a part thereof, and in which there is shown by way of illustration, and not limitation, preferred embodiments of the invention. These embodiments do not represent the full scope of the invention. Rather, reference should therefore be made to the claims herein for interpreting the scope of the invention.

### BRIEF DESCRIPTION OF THE DRAWINGS

While the specification concludes with claims which particularly point out and distinctly claim the present invention, it is believed that the present invention will be better understood from the following description of preferred embodiments, taken in conjunction with the accompanying drawings, in which like reference numerals identify identical elements and wherein:

FIG. 1 is a perspective view of a first embodiment of the present invention, showing the location of the thermal wrap and its thermal elements relative to the spine;

FIG. 2. is a perspective view of the thermal wrap shown in FIG. 1, showing the thermal wrap draped over a user's shoulders;

FIG. 3 is a plan view of the embodiment of the thermal wrap shown in FIG. 1;

FIG. 4 is a sectioned elevation of the embodiment of FIG. 3, taken along section line 4-4 of FIG. 3, showing an individual thermal element embedded within the thermal wrap;

FIG. 5 is a plan view of a second embodiment of the thermal wrap of the present invention;

FIG. 6 is a plan view of a third embodiment of the thermal wrap of the present invention;

FIG. 7 is a plan view of a fourth embodiment of the thermal wrap of the present invention;

FIG. 7A is a plan view of a buckle shown in the embodiment of FIG. 7;

FIG. 7B is a bottom elevation of the buckle shown in FIG. 7A;

FIG. 8 is a plan view of a fifth embodiment of the thermal wrap of the present invention; and

FIG. 8A is a partial bottom plan view of the thermal wrap of FIG. 8, showing the pocket detail.

### DETAILED DESCRIPTION OF THE INVENTION

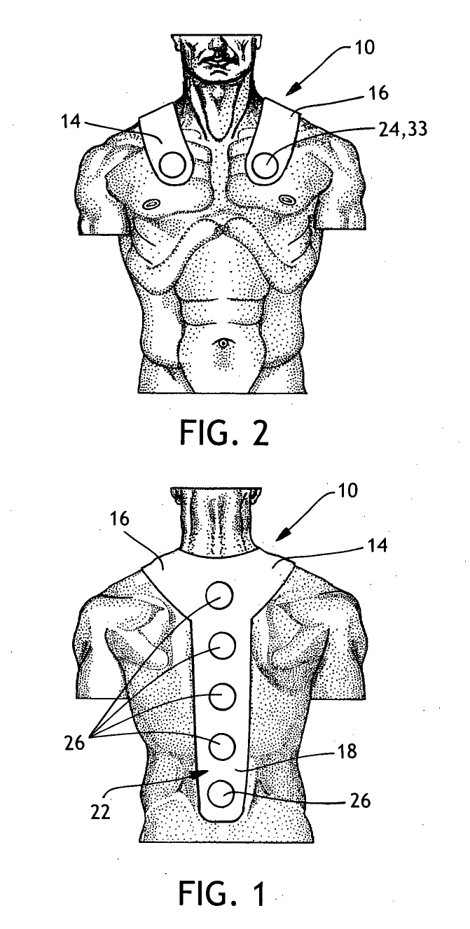

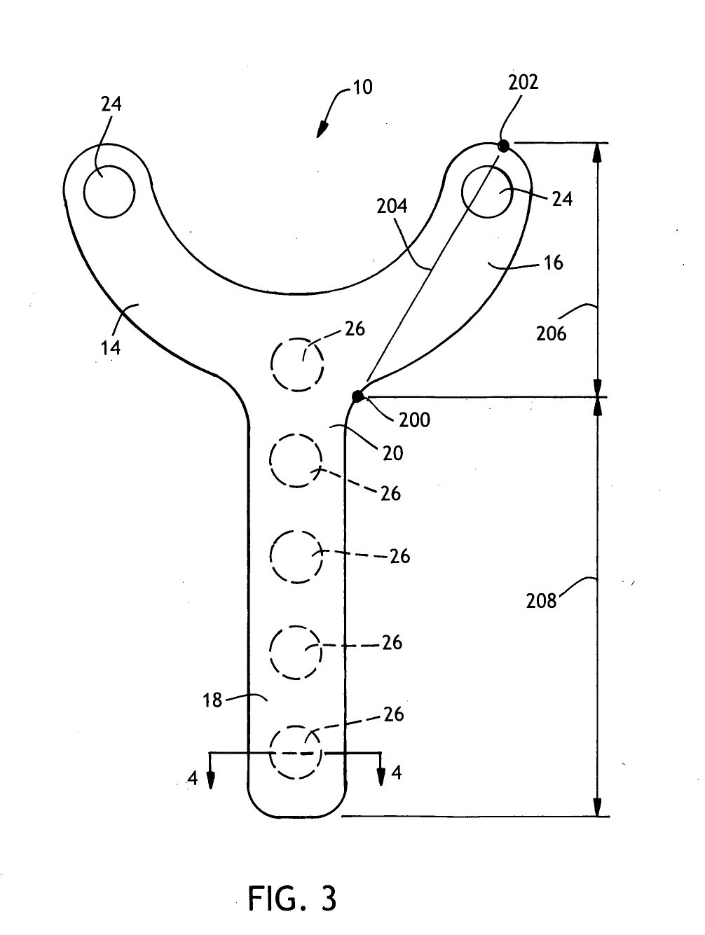

Referring now to the drawings, and more particularly to FIGS. 1-3, there is shown a first embodiment of the present invention which provides a thermal wrap being generally indicated as 10. Thermal wrap 10 is constructed using a flexible material 12 which, when planar or flattened, may have the appearance of a Y-shape (FIG. 3). Wrap 10 has a first wing portion 14, a second wing portion 16, and an elongated central body portion 18 therebetween. Wing portions 14 and 16 may have either curved edges as depicted in FIG. 3, straight edges, or even decorative edges (e.g. scalloped). Wrap 10 has a body facing side 20 and an opposing outer side 22.

The thermal wrap 10 is substantially symmetric about the longitudinal axis of the central body portion 18. The length of the central body portion 18 is about the length of distance 208 as seen in FIG. 3. Desirably, the length between point 200 and point 202 on wing portion 16 (or 14) may be less than the distance 208. Further, and the length of the wing portions along the longitudinal axis of central body portion 18 is generally referenced as length 206. Desirably, length 208 is greater than length 206. However, it is contemplated that the length 208 may be about 0.2 times the length 206 or greater. In the alternative, length 208 may be about 0.33 times the length 206 or greater. In a further alternative, length 208 may be about 0.5 times the length 206 or greater. In a further alternative, length 208 may be about 0.75 times the length 206 or greater. In a further alternative, length 208 may be about same as length 206 or greater. This may be applied to all the embodiments of the present invention shown in FIGS. 1-8.

When wrap 10 is worn, wing portions 14 and 16 desirably extend over the shoulders of the wearer onto the upper chest of the wearer (which may be a man or a woman), as depicted in FIG. 2. Central body portion 18 is located on the upper back, neck, and spine of the wearer, as depicted in FIG. 1. The central body portion may cover all or part of the thoracic region of the spine, and may further extend to cover all or part of the lumbar and possibly, the pelvic region of the spine. The central body portion may be configured to end in the region of a particular vertebra in the spine.

Thermal wrap 10 may have one or more individual thermal elements 26 embedded therein. Thermal elements 26 are positioned within the central body portion 18, and optionally within the wings 14 and 16. When wrap 10 is positioned on the user, thermal elements 26 may correspond to major muscle groups in the user's back and optionally, shoulders. The number, size, and shape of the thermal elements may vary from that depicted in the Figures, and should not be limited to the examples shown. For example, there may be several small thermal elements 26 instead of a few larger elements. In addition, the thermal elements may vary in size and/or shape depending on where they are intended to be positioned on the body. Thermal elements could individually have a decorative shape, or together as a group, take on an aesthetic pattern. For example, thermal elements could be in the shape of butterflies, flowers, whorls, geometric shapes, etc.

Thermal elements 26 are preferably heating elements that include an exothermic compound referred to as chemistry 42. In one embodiment, chemistry 42 is a mixture of powdered iron, powdered activated charcoal, vermiculite, water, and salt. In another embodiment, chemistry 42 is a mixture of activated carbon, iron powder, water, sale, macromolecule, and Diatomite. In yet another embodiment, chemistry 42 is a mixture of activated carbon, iron powder, water, salt, macromolecule, and Vermiculate. Most any compound having an exothermic reaction upon exposure to air will work, particularly one based on an iron powder, providing that the compound does not produce enough heat to burn the user or create general discomfort.

Mixtures like chemistry 42 react when exposed to oxygen providing heat for up to several hours. Prior to use, the wrap 10 is enclosed within an oxygen impermeable package. To use wrap 10, it is removed from the oxygen impermeable package allowing oxygen to enter pockets 40 and react with chemistry 42 of thermal elements 26.

Thermal elements 26 may be constructed by thermoforming a base material 38 to form a pocket 40. Pocket 40 is then filled with chemistry 42. After filling pocket 40 with chemistry 42, cover material 44 is placed over pocket 40 and bonded to base material 38 around the periphery of pocket 40, for example, with a heat seal. The bonding will encapsulate chemistry 42. Small apertures may be placed in base material 38 and/or cover material 44 to allow oxygen to reach chemistry 42 if material 12 is of inadequate porosity for an exothermic reaction to occur.

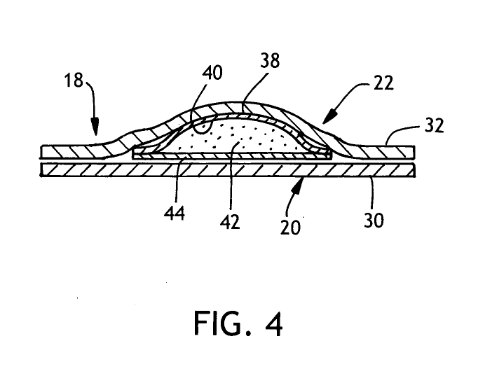

Thermal elements 26 may be enclosed entirely within two or more sheets 30, 32 of flexible material 12 as depicted in FIG. 4. In this case, material 12 is breathable enough to allow oxygen to reach the thermal elements in order that the warming reaction can occur. Material 12 may have a porosity that would allow this to occur, or apertures may be placed in material 12, at least in the regions covering the thermal elements 26. Alternatively, thermal elements 26 may be attached to either the body-facing side 20 or opposing outer side 22. In this case, the flexible material may be constructed from a single or multiple sheets of material. Regardless, thermal elements 26 are desirably spaced apart from one another so the wrap remains relatively flexible. Of course, the sheet 30 or 32 may be used to create pocket 40 to contain the chemistry 42, omitting the need for base material 38 and/or cover material 44, depending on whether there are two plies of material 12 between which the chemistry 42 may be contained.

Base material 38 and cover material 44 may be made of any number of materials capable of containing chemistry 42 and limiting oxygen flow into pocket 40. One such non-limiting example of material 12 is a 42 gram per square meter polypropylene spunbond nonwoven which has been extrusion coated with low density polyethylene and/or ethyl vinyl acetate (EVA) at a thickness of 50 to 75 microns.

Desirably, the thermal elements 26 resemble the size of stones used in typical spa treatments. For example, a thermal element 26 may be an ovoid having a length of about 200 mm, a width of about 145 mm, and a thickness of about 85 mm. However, the exact size and shape of the thermal elements may vary greatly, and it is contemplated that any shape would be possible. It may be desirable to provide thermal elements 26 that range anywhere from about 2.54 cm in diameter to about 10 cm in diameter. The thermal element thickness may range from about 5 mm to about 254 mm. Thermal elements having a greater mass and thickness may provide a longer exothermic reaction than a thermal element having less relative mass and/or less relative thickness.

Flexible material 12 may be selected from any number of suitable materials including but not limited to: wovens, knits, films, foams, and nonwovens. The nonwovens include spunbond, spunbond-meltblown, spunbond, carded, meltblown, hydroentangled, through-air bonded, air laid, and wet laid. These materials may be made from natural fibers such as cotton, wool, linen, tencel, hemp, and the like; or manmade materials such as: polypropylene, polyester, nylon, polyethylene, metallocene catalyst polyethylene, and the like. Desirably, a nonwoven is used for material 12, having a basis weight light enough to allow oxygen to permeate and activate the thermal element 26. One particular non-limiting example of material 12 is a 55 OSY basis weight spunlace.

The material basis weight and/or type may vary depending upon the desired effect of the thermal element 26. The faster oxygen that is able to reach the chemistry 42, the faster the thermal element 26 will exhibit an exothermic reaction. Of course, the faster an exothermic reaction occurs for a given amount of chemistry 42, the faster the total exothermic reaction occurs. It is most desirable that the exothermic reaction in one or more of the thermal elements 26 be detectable within about 5 minutes of exposure to a catalyst such as air or oxygen. This time may be shorter or longer, as desired.

During use, thermal wrap 10 may be draped over the shoulders of the wearer. Optionally, first and second wing portions 14 and 16 may have weights 24 placed in their distal ends. Such weights 24 are located on the upper chest of the wearer as depicted in FIG. 2 and serve to counterbalance the weight of thermal elements 26 which are located substantially on the back of the wear during use. In a preferred embodiment of the present invention, weights 24 may be about equal or greater than about one-half of the weight located in central body portion 18 (sum of thermal elements 26 and material forming central body portion 18).

Any number of suitable materials could be used to provide the counterweight needed. Weights are preferably placed on the first and second wing portions with an adhesive, such as a hot melt adhesive, or the like. In the alternative, the weights may be disposed in pockets formed between layers of the flexible material 12.

It is further contemplated that in lieu of, or in addition to the weights 24, there may be a body adhesive located on body-facing surface of wings 14 and 16. Though not depicted, the body adhesive may be configured into swirls, a continuous film, strips, or dots. For example, the body adhesive may be configured into a patch 33 located on the body-facing surface 20, as shown in FIG. 3. It is further contemplated that the body adhesive may be placed on all or a portion of the body-facing surface 20 of the central body portion 18 and/or wing portion 14 and 16, for example, at distal end 19 of the central body portion and on each wing portion 14 and 16.

The body adhesive may be a hydrogel, two-faced tape, or other type of skin-friendly adhesives such as that used to construct bandages or tapes for the fashion industry or medical use. One non-limiting example of a body adhesive is DERMA-TAK 34-546B, manufactured by National Starch, headquartered in Bridgewater, N.J. The body adhesive may be protected by a removable release paper until ready for use.

Thermal wrap 10 may further include at least one foamed polymer strip attached to each of first wing portion 14 and second wing portion 16 on body-facing side 20 (not shown). Foamed polymer strips could provide increased friction between wrap 10 and the wearer in order to reduce slipping or relative movement between wrap 10 and the wearer during use.

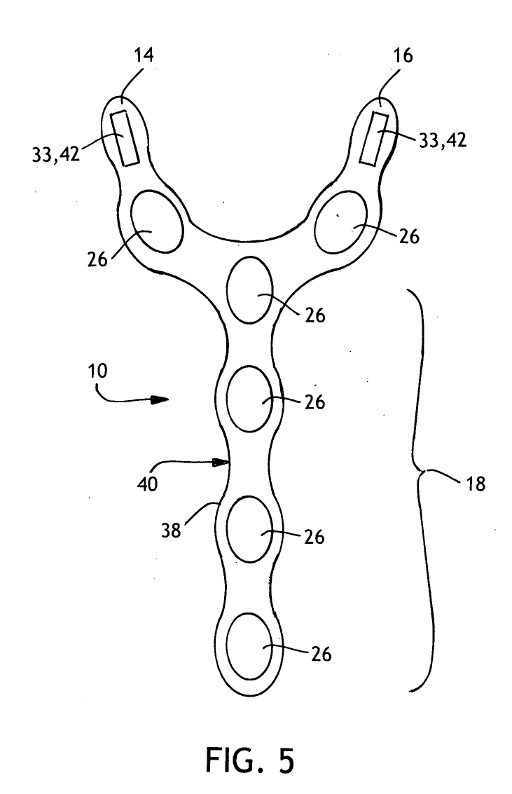

Various other embodiments as shown in FIGS. 5-8 demonstrate how the wrap 10 may be more securely positioned on the body, or how thermal elements may be attached to the flexible material 12. Concepts from these embodiments may be interchanged or added to one or more of the embodiments shown in FIGS. 1-8, and these definite examples are not meant to limit the scope of the present invention.

Referring now to FIG. 5, there is another embodiment of the present invention showing a thermal wrap 10 constructed from a single layer of material 12 (as described above) onto which an array of thermal elements 26 (also, described above) are attached. Desirably, material 12 is shaped into an overall Y-shape with minimized surface area so that the thermal elements 26 appear to be more prominent with respect to the overall structure. Thus, between each thermal element in the central body portion 18, material 12 may have an edge 38 which forms a concave portion 40.

Attachment between the thermal elements 26 and the surface 20 of material 12 may be made with an adhesive such as hot melt, two-faced tape, and the like. Other methods of attachment, such as hook and loop, are possible.

Because thermal elements 26 are visible, they may include printed or dyed indicia such as an aesthetic or informational feature (e.g. a desired pattern or wording). For example, the thermal elements 26 may be printed with an ink that makes it resemble a river stone. Other aesthetic features are possible, e.g. colored substrate.

As with the previous embodiment, a body adhesive 42 may be placed onto the wing portions 14 and 16, or the central body portion 18.

Prior to use, the wrap 10 is enclosed within an oxygen impermeable package. To use wrap 10, it is removed from the oxygen impermeable package allowing oxygen to enter pockets 40 and react with chemistry 42 of thermal elements 26.

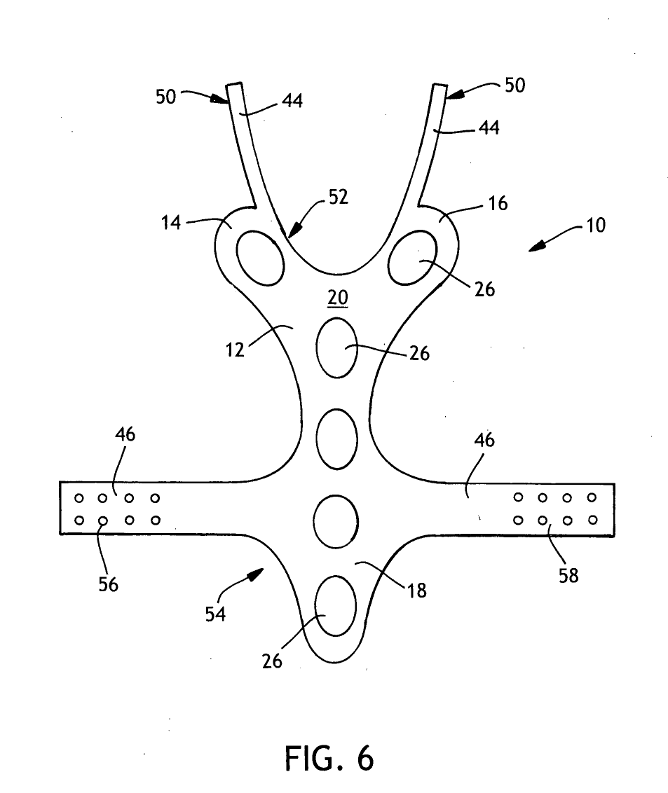

Referring now to FIG. 6, there is yet another embodiment of the present invention showing a thermal wrap 10 constructed from a single layer of material 12 (as described above) onto which an array of thermal elements 26 (also, described above) are attached. This embodiment may include collar sections 44 extending from each of the wing portions 14 and 16. In addition, this embodiment may include an optional belt member such as belt portions 46 extending laterally from the central body portion 18.

Collar sections may be separate or integral extensions of the wing portions 14 and 16. Collar sections 44 may be long enough so that they may be tied to together for fastening about the neck of the user. In an alternative embodiment, the collar sections 44 may include corresponding hook and loop fasteners at each distal end 50 (not shown) to provide the fastening about the neck. Other methods of fastening include hook and eye, snaps, adhesive, buttons, clips, and the like. Desirably, the collar sections 44 form a U-shape defined by an inner edge 52 of the wing portions 14 and 16.

So that the thermal wrap 10 remains against the body as a person moves about, it may be desirable to include the belt member such as belt portions 46. Belt portions may be separately or integrally attached to the central body portion 18. The location of belt portions may be about where a person's natural waist would be. This may be different for men and woman, and it may thus be desirable to make different sizes or gender specific versions of thermal wrap 10. Belt portions 46 may be made from the same material as material 12, or may be an alternative material. Desirably, there are radiused portions 54 located at the junction between the central body portion 18 and belt portions 46 to possibly improve comfort and durability of the thermal wrap 10. Belt portions 46 may be fastened several methods including hook and loop, adhesive, snaps, hook and eye, button(s), and the like. It is further contemplated that the belt portions may be long enough to tie together. Desirably, the fit is one that is adjustable, such as that obtainable with a hook and loop as shown. A hook region 56 and loop region 58 may fully or partially overlap to form a belt, as desired.

As previously described, attachment between the thermal elements 26 and the surface 20 of material 12 may be made with adhesive such as two-faced tape, hot melt, and the like. Other methods of attachment such as hook and loop, are possible. In addition, because thermal elements 26 may be visible, they may include an aesthetic feature such as a desired color or pattern.

Prior to use, the wrap 10 is enclosed within an oxygen impermeable package. To use wrap 10, it is removed from the oxygen impermeable package allowing oxygen to enter pockets 40 and react with chemistry 42 of thermal elements 26.

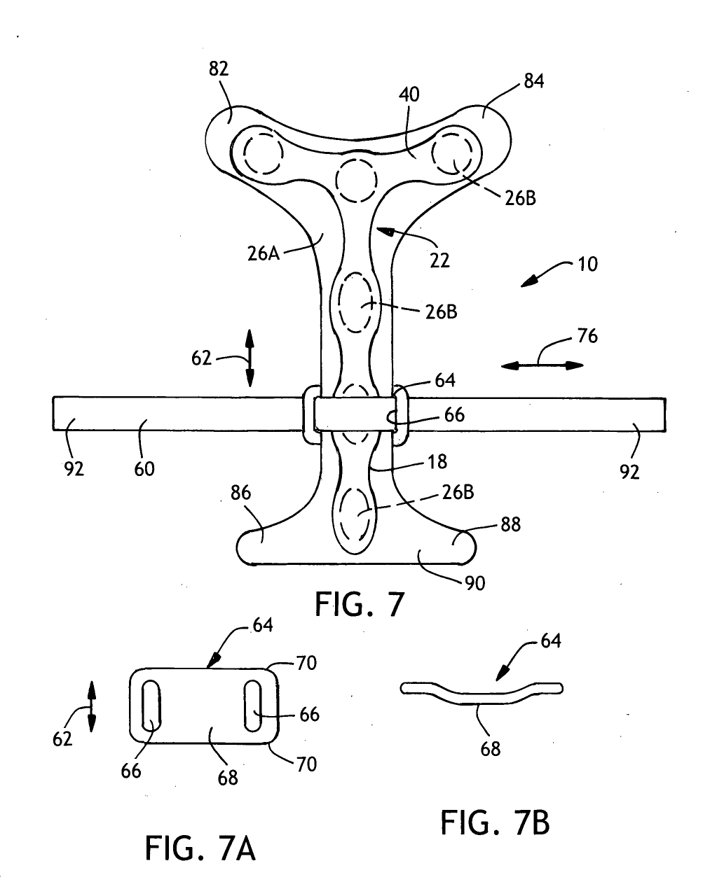

Referring now to FIG. 7, there is yet another embodiment of the present invention showing a thermal wrap 10 that uses a body adhesive and an optional belt member such as belt 60 to keep the thermal wrap 10 in contact with the user's back even with significant movement. Generally, the thermal wrap 10 has a belt 60 that can slide along the central body portion 18 in the direction 62, which substantially coincides with parallel with the longitudinal axis of central body portion 18.

The overall shape of thermal wrap 10 may be a capital I-shape, as depicted, or the T-shape as previously described. Desirably, the body-facing surface opposite that of surface 22 is effectively covered, either continuously or discontinuously with a body-adhesive, as described previously. The body adhesive serves to adhere the thermal wrap 10 to the user's body so that it will not fall or gap away from the user's body during movement. The body adhesive may be applied as a film, dots, swirls, stripes, or the like.

Referring still to FIG. 7, thermal wrap 10 may include wing portions such as first wing portion 82 and a second wing portion 84. Different from the wing portions 12 and 14, wing portions 82 and 84 may not extend onto the shoulder or above the clavicle of the user. Desirably, wing portions 82 and 84 are placed on the upper back over the shoulder blades of the user so that thermal elements may be applied in those regions. Because body adhesive is used, there is no need for the wing portions to help with attachment of the thermal wrap 10 to the body as described in the previous embodiments.

Thermal wrap 10 may further include tail portions, such as a first tail portion 86 and a second tail portion 88. Tail portions 86 and 88 serve as handles so users can more easily position the thermal wrap along the desired portion of the body. Tail portions 86 and 88 may extend laterally outward from the central body portion 18, and rest along the flank or hips of a user, depending on the length of central body portion 18. Desirably, tail portions adhere to the user's skin so that they are easily found for adjustment or removal.

The optional belt 60 may be selectively attached to the thermal wrap 10 with a buckle member 64, as shown by itself in FIG. 7A. Desirably, buckle member 64 may be constructed from metal, plastic, fiberglass, or any other heat conductive material that can be formed into a relatively thin, flat or almost flat piece having a pair of slots 66 to accommodate the belt 60. For example, buckle member 64 may be formed by injection molding a plastic material into a plate having a slight curvature aligned to generally conform with a user's spine. Desirably, buckle member 64 exhibits some flexure and has rounded edges and corners 70 for better comfort. It is contemplated that buckle member 64 may be generally rectangular or oval in shape. Further, it is contemplated that buckle member 64 may be made from other materials such as wood, paper, and other various composites.

Belt 60 may be a strip of flexible material obtained from a plastic sheet, extruded plastic, web, woven or non-woven material. Belt 60 is long enough to wrap around a user's torso, and is selectively fastened with a fastener such as hook-and-loop, snaps, hook-and-eye, adhesives, pins, buckles, and the like. In the alternative, belt 60 is fastened by tying or looping back into buckle member 64. The method of fastening the belt 60 about the user's torso is not critical to the invention. Belt 60 may selectively slide in direction 76, a direction perpendicular to direction 62.

Thermal elements 26 may be combined into an elongated strip or a T-shaped unit as shown in FIG. 7. For instance, the thermal elements may be distinct and separate as described previously, or they may be combined into a single thermal unit 26A having a series of thermal foci 26B. Each thermal focus 26B may be sandwiched between T-shaped layers of material 40 and 42 as described for FIG. 4. In the alternative, the thermal foci 26B may be sandwiched between the flexible I-Shaped substrate 90 and a T-Shaped layer of material 40 as depicted. Materials 40, 42 and 90 are the same as described previously regarding material 12. There may be a continuous amount of chemistry 42 between each thermal element focus 26B.

To assemble the thermal wrap 10 with optional belt 60, as depicted in FIG. 7, the buckle 64 is placed against the body-facing surface 20 of the thermal wrap body so that apertures 66 are visible. Opposite ends 92 of belt 60 are threaded through apertures 66.

In an alternative embodiment, buckle 64 is placed against the surface 22 of the thermal wrap body so that apertures 66 are visible. In a further alternative embodiment, the body adhesive is placed on surface 22 only, and the material 40 operates as a body facing surface.

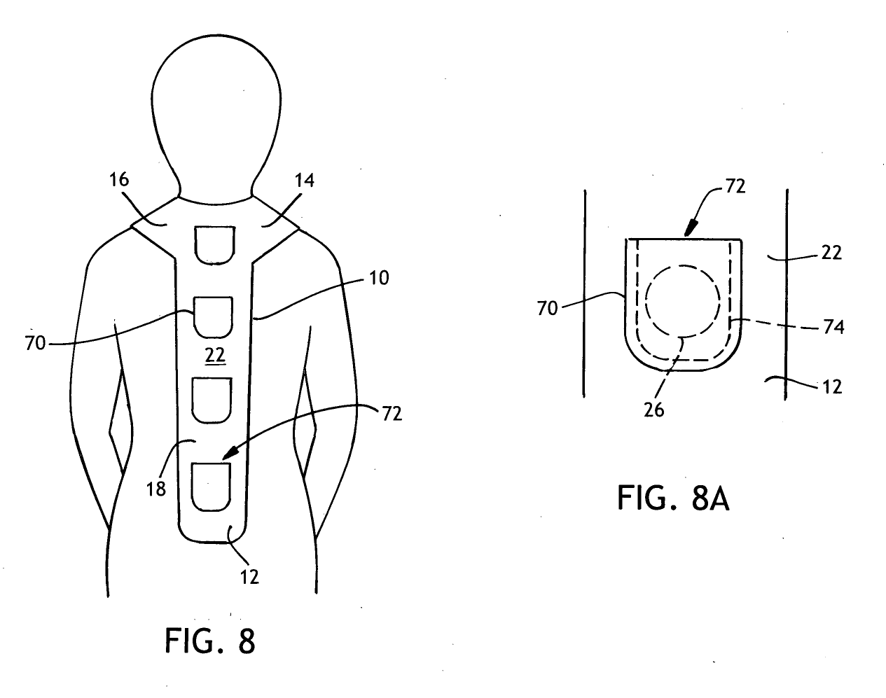

Referring now to FIGS. 8 and 8A, shown is yet another embodiment of the thermal wrap 10 having pockets, such as patch pockets 70 for containing thermal elements 26. Each pocket 70 is formed from at least a single sheet of material, such as material 12, and attached to the surface 22 (or 20) of the thermal wrap 10. It is contemplated that the pocket could be formed from a breathable material (allowing the thermal reaction to occur in thermal elements 26) such as mesh, or a breathable translucent material. Breathability may be inherent to such a material, or may be due to apertures placed therein.

The pocket is attached to the material 12 defining the body of thermal wrap 10 by stitching, stapling, adhesive, ultrasonic bonding, thermal bonding, and the like. As seen in FIG. 8A, a seam 74 may surround all sides of the pocket 70 with the exception of an open edge such as top edge 72. In the alternative, pocket 70 may be sealed on all sides and have a slot so that a thermal element 26 may be placed therein.

Desirably, the pockets 70 are spaced apart along the central body region 18, but may be included in the wing portions 14 and 16 also. Each pocket may be shaped like a patch pocket having an open edge 72. Other shapes may be used as long as they are large enough to accommodate a single thermal element 26.

Thermal elements 26 and/or pockets 70 may have aesthetic features as described previously, or may have instructions printed or placed thereon so that the user understands where to place the thermal element 26 on the thermal wrap 10.

Prior to use, the thermal elements 26 are enclosed within an oxygen impermeable package. Prior to use, each thermal element 26 is removed from the oxygen impermeable package and placed into a pocket 70 as the reaction with oxygen occurs.

While particular embodiments of the present invention have been illustrated and described, it will be obvious to those skilled in the art that various changes and modifications may be made without departing from the spirit and scope of the invention, and it is intended to cover in the appended claims all such modifications. In particular, portions of one embodiment such as a collar portion, belt portion, pockets, weights, body adhesives, etc. may be interchanged or added to features of another described embodiment. Further, the thermal wrap 10 may be sold as a kit that includes instructions, candles, tea, massage oil, recorded music, perfume, and multiple and separate parts that are combined by the user to construct the thermal wrap 10.

1. A thermal wrap for a user's spine comprising:
an elongated piece of flexible material defining a central body portion having a first end and a second end;
a first wing portion separated from a second wing portion, each extending outwardly from the first end;
a first thermal element having an exothermic chemistry, the first thermal element disposed on the central body portion;
wherein the central body portion and the first thermal element are adapted to be disposed on the user's spine during use.

2. The thermal wrap of claim 1 further comprising counterweights attached to said first and second wing portions.

3. The thermal wrap of claim 1 further comprising at least one strip of foamed polymer attached to each of said first and second wing portions on a body-facing side thereof, said at least one strip of foamed polymer reducing slip between the piece of flexible material and a user's body.

4. The thermal wrap of claim 1 further comprising collar portions extending from each of the first and second wing portions for securing the thermal wrap about a user's neck.

5. The thermal wrap of claim 1 further comprising thermal elements located in the first and second wing portions.

6. The thermal wrap of claim 1 further comprising a belt member extending outwardly from the central body portion for securing the thermal wrap about a user's torso.

7. The thermal wrap of claim 1 further comprising a pocket on the central body portion in which the first thermal element may be disposed.

8. The thermal wrap of claim 7 wherein the pocket has an opening for selective placement of the first thermal element.

9. The thermal wrap of claim 8 wherein the pocket has apertures therein.

10. The thermal wrap of claim 8 wherein the pocket comprises a mesh material.

11. The thermal wrap of claim 1 wherein a body-facing surface of the thermal wrap has a body adhesive disposed thereon.

12. The thermal wrap of claim 11 wherein the body adhesive is a continuous film.

13. The thermal wrap of claim 11 wherein the body adhesive covers only a portion of the area defined by the central body portion, the first wing portion, and the second wing portion.

14. The thermal wrap of claim 11 wherein the body adhesive comprises a hydrogel.

15. The thermal wrap of claim 1 further comprising at least one additional thermal element spaced apart from the first thermal element on the central body portion.

16. The thermal wrap of claim 1 further comprising a first tail portion and a second tail portion extending from the second end of the central body portion.

17. A kit for providing heat to a user's spine comprising:
an elongated piece of flexible material defining a central body portion having a first end and a second end, with a first wing portion separated from a second wing portion extending outwardly from the first end; and
a thermal element having an exothermic chemistry, the thermal element adapted to be disposed on the central body portion;
wherein the central body portion and the thermal element are adapted to be disposed on the user's spine during use.

18. The kit of claim 17 further comprising a pocket located on the central body portion for selectively containing the thermal element.

19. The kit of claim 17 wherein the thermal element comprises a plurality of thermal foci.

20. The kit of claim 17 further comprising an adjustable belt member.
